(19) United States
(12) Patent Application Publication
(10) Pub. No.: US 2026/0265217 A1
(43) Pub. Date: Sep. 10, 2026

(54) A METHOD FOR THE PREPARATION OF ANTHRANILIC DIAMIDES

(71) Applicant: PI INDUSTRIES LTD., Rajasthan Udaipur (IN)

(72) Inventors: Nitin MALVIYA, Rajasthan Udaipur (IN); Jigarkumar Harkishandas SHAH, Rajasthan Udaipur (IN); Vipender SINGH, Rajasthan Udaipur (IN); Alexander G.M. KLAUSENER, Pulheim (DE)

(21) Appl. No.: 19/166,619

(22) PCT Filed: Mar. 21, 2024
(86) PCT No.: PCT/IN2024/050290

§ 371 Date: Sep. 18, 2025

(30) Foreign Application Priority Data
Mar. 22, 2023 (IN) 202311019990

(51) Int. Cl. C07D 401/04; C07D 413/14

(52) U.S. Cl. CPC C07D 401/04; C07D 413/14

(57) ABSTRACT

This patent application discloses novel methods for preparing anthranilic diamides, their intermediates, N-oxides and salts thereof.

### FIELD OF THE INVENTION

The present invention relates to a method for the preparation of substituted 1-pyridinylpyrazole-5-carboxylic acids of Formula 3, its intermediates, N-oxides or salts thereof.

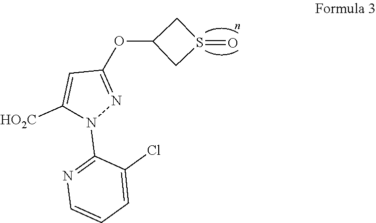

wherein n is as described herein.

Further, the present invention relates to a method for the preparation of anthranilic diamide compounds of Formula 1, its intermediates, N-oxides or salts thereof.

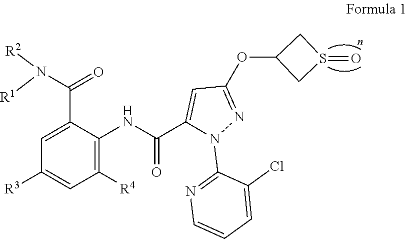

wherein, n, R¹, R², R³ and R⁴ are as defined herein.

### BACKGROUND OF THE INVENTION

1-Pyridinylpyrazole-5-carboxylic acids are known to be important intermediates in the agrochemical industry, e. g. for the synthesis of anthranilic diamides which are useful to protect crops against harmful pests. Several methods have been disclosed by which these intermediates can be obtained.

WO 2019150220 discloses anthranilic diamides of Formula (I), and their use as insecticides,

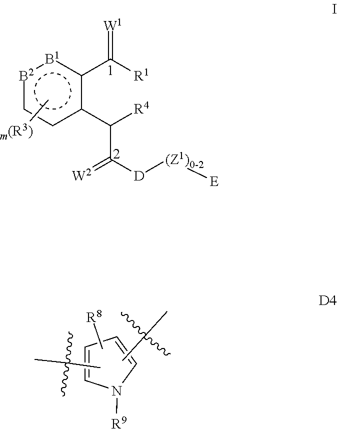

wherein,
D represents D4; and Z¹ is independently a direct bond or CR⁶R⁷ or NR^{c} or O or S(O)₀₋₂; and
E represents 4 membered heterocycles. It also provides a process for preparing the said compounds of Formula (I).

WO 2022254395 discloses a process for preparing anthranilic diamides of Formula (I),

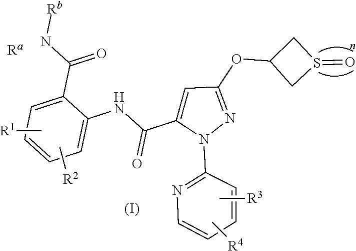

wherein,
R^{a}, R^{b}, R¹, R², R³, R⁴ and n are as defined in the description.

The process described in the prior art has shortcomings such as a longer synthetic route, use of additional agents such as halogenating agents, oxidizing agents and catalysts, thus making the process economically unviable in a way that it is not suitable for a commercial scale preparation of the anthranilic diamide compounds and intermediates thereof. Furthermore, processes described in the prior art, namely in WO 2019150220 and WO 2022254395, are involving Mitsunobu reactions for preparing the etherified product from ethyl 1-(3-chloropyridin-2-yl)-3-hydroxy-1H-pyrazole-5-carboxylate and thietane-3-ol or 3-hydroxy-thietane-1,1-dioxide. Thus, these processes disclosed in the prior art have shortcomings such as application of moisture-free conditions, dry solvents, and expensive reagents such as diethylazadicarboxylate (DEAD) or diisopropylazadicarboxylate (DIAD) and triphenylphosphine, required for performing the Mitsunobu reactions.

Therefore, there is a need for a scalable method that is simple, efficient, and economically viable and that overcomes at least one of the shortcomings associated with the known methods.

The present invention provides a novel method for preparing the anthranilic diamides of Formula 1, its intermediates, N-oxides or salts thereof, preferably compounds of Formula 3, its intermediates, N-oxides or salts thereof, with good yield on a commercial scale, wherein the described method is shorter, efficient, avoiding the use of additional agents such as halogenating agents, oxidizing agents, and specific catalysts as well as the Mitsunobu reaction as such.

### Objective of the Present Invention

The objective of the present invention is to provide a simple and economically viable method for the preparation of compounds of Formula 3, its intermediates, N-oxides or salts thereof, on a commercial scale.

Another objective of the present invention is to provide a simple, efficient, and economically viable method for preparing anthranilic diamide compounds of Formula 1, its intermediates, N-oxides or salts thereof, on a commercial scale.

The present invention provides a solution to these objectives by offering high yield and economical method that allows the preparation of anthranilic diamide compounds of Formula 1 and/or key intermediates, namely compounds of Formula 3, to prepare such anthranilic diamide compounds of Formula 1, by overcoming at least one of the shortcomings of the processes described in the prior art.

### SUMMARY OF THE INVENTION

The objectives of the present invention are achieved by providing a novel, efficient and economically viable method for preparing compounds of Formula 3, its intermediates, N-oxides or salts thereof,

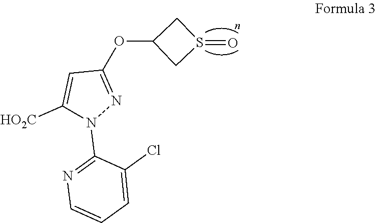

wherein n is an integer selected from 0-2,
comprising the steps of:
   a. preparing a compound of Formula 5 from a compound of Formula 6 in the presence of a suitable reagent and a suitable solvent (A);

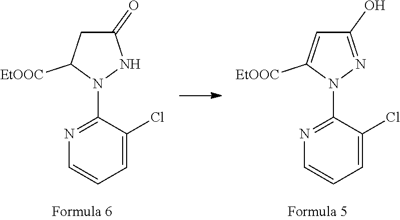 [SMILES: CCOC(=O)C1CC(=O)NN1c1ncccc1Cl ; CCOC(=O)c1cc(O)nn1-c1ncccc1Cl]

b. reacting the compound of Formula 5 with a compound of Formula 10 in the presence of a suitable base (I), a suitable solvent (B), and an optional phase transfer catalyst, to obtain a compound of Formula 4, wherein the compound of Formula 4 is optionally isolated; and

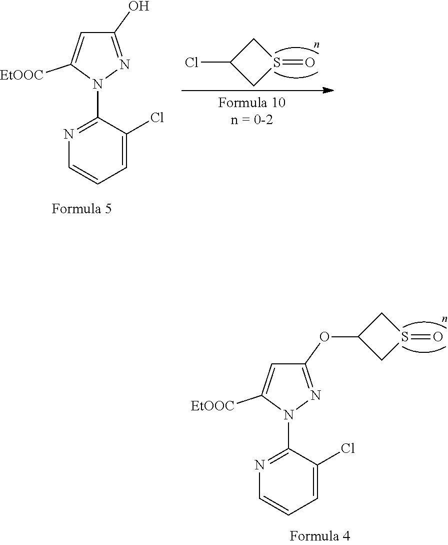

c. hydrolyzing the compound of Formula 4 in the presence of a hydrolyzing agent to obtain a compound of Formula 3,

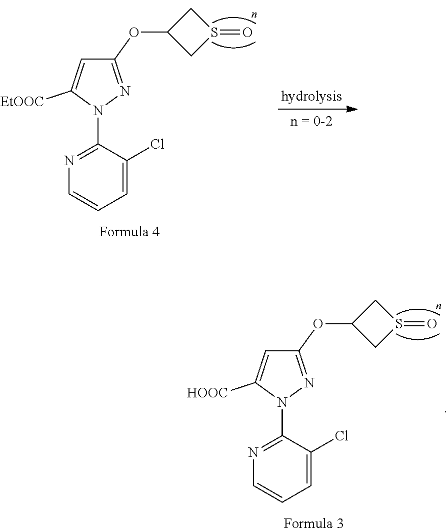

The present invention also provides a method for preparing anthranilic diamide compounds of Formula 1, its intermediates, N-oxides or salts thereof,

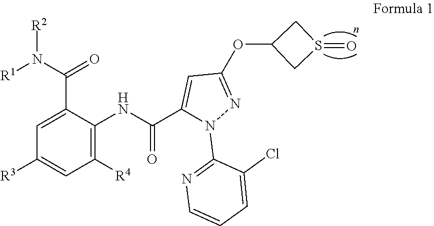

wherein,
R¹ is selected from hydrogen or C₁-C₆ alkyl;
R² is selected from C₁-C₆ alkyl or C₃-C₆ cycloalkyl, wherein said C₁-C₆ alkyl and C₃-C₆ cycloalkyl is optionally substituted with one or more substituents selected from a group consisting of halogen and C₃-C₆ cycloalkyl;
R³ is selected from hydrogen, halogen or cyano;
R⁴ is selected from halogen, C₁-C₆ alkyl, C₁-C₆ alkoxy or C₁-C₆ haloalkyl; and
n is an integer selected from 0-2, comprising the steps of:
   a. preparing a compound of Formula 5 from a compound of Formula 6 in the presence of a suitable reagent and a suitable solvent (A);

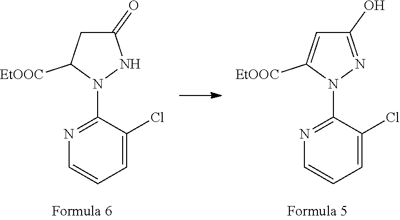 [SMILES: CCOC(=O)C1CC(=O)NN1c1ncccc1Cl ; CCOC(=O)c1cc(O)nn1-c1ncccc1Cl]

b. reacting the compound of Formula 5 with a compound of Formula 10 in the presence of a suitable base (I), a suitable solvent (B), and an optional phase transfer catalyst, to obtain a compound of Formula 4, wherein the compound of Formula 4 is optionally isolated;

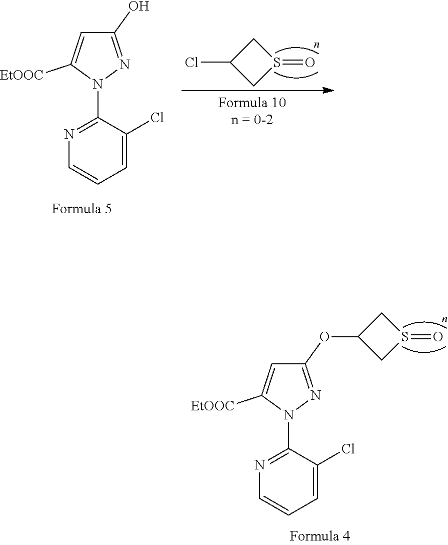

c. hydrolyzing the compound of Formula 4 in the presence of a hydrolyzing agent to obtain a compound of Formula 3;

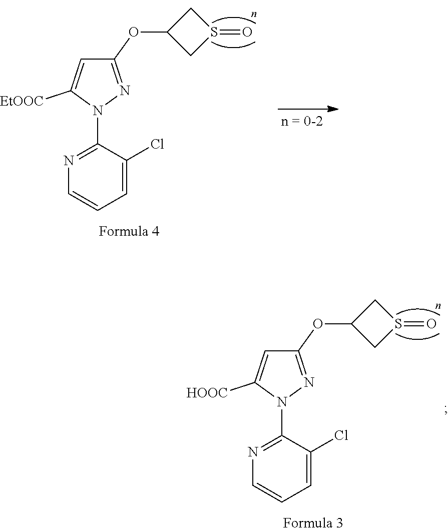

and
d. reacting the compound of Formula 3 with an acid chloride in a suitable solvent (C) and a suitable base (II) to obtain anthranilic diamide compound of Formula 1, its intermediates, N-oxides or salts thereof.

### DETAILED DESCRIPTION OF THE PRESENT INVENTION

As used herein, the terms “comprises”, “comprising”, “includes”, “including”, or any other variation thereof, are intended to cover a non-exclusive inclusion, subject to any limitation explicitly indicated. For example, process or method that comprises a list of elements is not necessarily limited to only those elements but may include other elements not expressly listed or inherent to process or method.

Also, the indefinite articles “a” and “an” preceding an element or component of the present invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

The compounds of the present disclosure may be present either in pure form or as mixtures of different possible isomeric forms such as stereoisomers or constitutional isomers. The various stereoisomers include enantiomers, diastereomers, chiral isomers, atropisomers, conformers, rotamers, tautomers, optical isomers, polymorphs, and geometric isomers. Any desired mixtures of these isomers fall within the scope of the claims of the present disclosure. One skilled in the art will appreciate that one stereoisomer may be more active and/or may exhibit beneficial effects when enriched relative to the other isomer(s) or when separated from the other isomer(s). Additionally, the person skilled in the art knows processes or methods or technology to separate, enrich, and/or to selectively prepare said isomers.

The compounds of the present disclosure may be present in the form of N-oxides or salts. The compounds of the present invention may be an acid addition or base addition salt. The acid addition salt includes inorganic or organic acid preferably hydrochloric acid, trifluoroacetic acid, methanesulfonic acid, p-toluenesulfonic acid. The base addition salt includes inorganic or organic base preferably alkali metal or alkaline earth metal salt.

The term “C₁-C₆ alkyl” used in the present invention refers to a linear or branched alkyl with 1 to 6 carbon atoms, may be optionally substituted by one or more substituents. Examples of C₁-C₆ alkyl includes but not limited to methyl, ethyl, n-propyl, isopropyl, n-butyl, isobutyl, sec-butyl, t-butyl, n-pentyl and n-hexyl.

The term “C₁-C₆ alkoxy” used in the present invention refers to a linear or branched alkyl with 1 to 6 carbon atoms attached via an oxygen linkage to the rest of the molecule, which may be optionally substituted by one or more substituents. Examples of C₁-C₆ alkoxy includes but not limited to methoxy, ethoxy, and the like.

The halogen used in the present invention refers to fluoro, chloro, bromo or iodo.

The term “C₁-C₆ haloalkyl” used in the present invention refers to a linear or a branched alkyl with 1 to 6 carbon atoms which is substituted with one or more halogen, may be optionally substituted by one or more substituents. Examples includes but not limited to trifluoromethyl, difluoromethyl, trifluoroethyl, perfluoroethyl.

The term “C₃-C₆ cycloalkyl” used in the present invention refers to a saturated non-aromatic carbocyclic ring with 3 to 6 carbon atoms, may be optionally substituted by one or more substituents. Examples of C₃-C₆ cycloalkyl includes but not limited to cyclopropyl, cyclobutyl, cyclopentyl, cyclohexyl.

In the context of the present invention, the term “optionally” or “optional” when used in reference to any element, to intermediates, reagents or conditions, including any method step, e.g., the isolation of intermediates; is intended to mean that the subject element is isolated, or alternatively is not isolated from the reaction mixture and directly used for the subsequent chemical reaction. Similarly, this definition is applied in case for reagents or reaction conditions as well.

The specification herein, the various features and advantageous details thereof are explained with reference to the non-limiting examples in the description. Descriptions of well-known components and processing techniques are omitted so as to not unnecessarily obscure the embodiments herein. The examples used herein are intended merely to facilitate an understanding of ways in which the specification herein may be practiced and to further enable those of skilled in the art to practice the specification herein. Accordingly, the examples should not be construed as limiting the scope of the specification herein.

The description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

Any discussion of documents, acts, materials, devices, articles and the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

Accordingly, the present invention provides a method for preparing a compound of Formula 3, its intermediates, N-oxides or salts thereof,

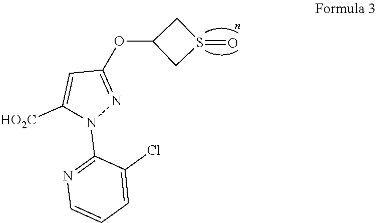

wherein n is an integer selected from 0-2,
comprising the steps of:
   a. preparing a compound of Formula 5 from a compound of Formula 6 in the presence of a suitable reagent and a suitable solvent (A);

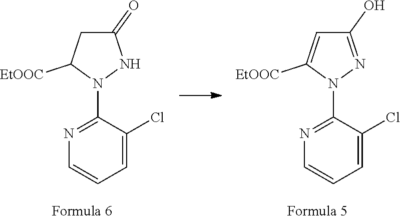 [SMILES: CCOC(=O)C1CC(=O)NN1c1ncccc1Cl ; CCOC(=O)c1cc(O)nn1-c1ncccc1Cl]

b. reacting the compound of Formula 5 with a compound of Formula 10 in the presence of a suitable base (I), a suitable solvent (B), and an optional phase transfer catalyst, to obtain a compound of Formula 4, wherein the compound of Formula 4 is optionally isolated; and

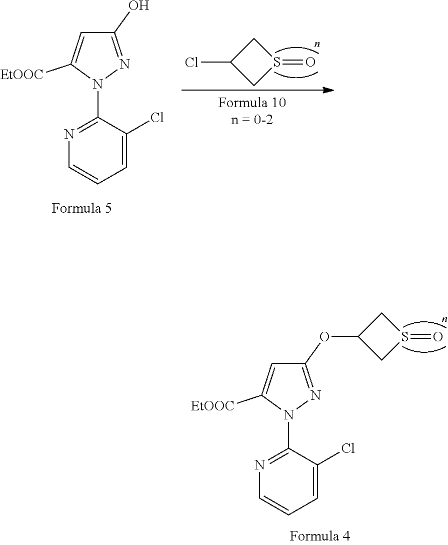

c. hydrolyzing the compound of Formula 4 in the presence of a hydrolyzing agent to obtain the compound of Formula 3.

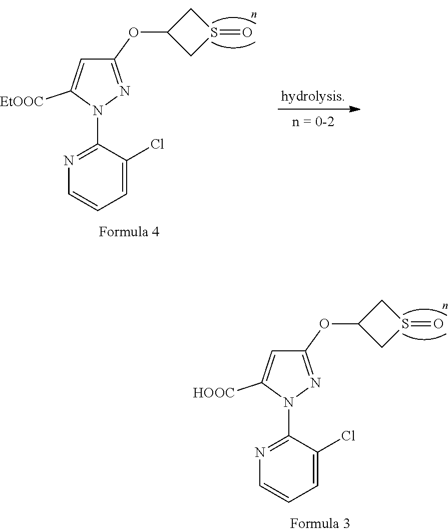

The present invention also provides a method for preparing anthranilic diamide of Formula 1, its intermediates, N-oxides or salts thereof,

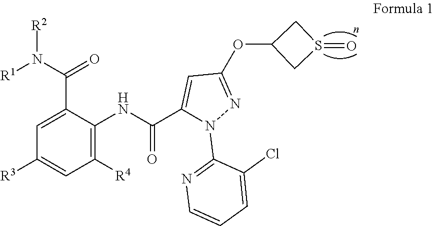

wherein,
   R¹ is selected from hydrogen or C₁-C₆ alkyl;
   R² is selected from C₁-C₆ alkyl or C₃-C₆ cycloalkyl, wherein said C₁-C₆ alkyl and C₃-C₆ cycloalkyl is optionally substituted with one or more substituents selected from a group consisting of halogen and C₃-C₆ cycloalkyl;
   R³ is selected from hydrogen, halogen or cyano;
   R⁴ is selected from halogen, C₁-C₆ alkyl, C₁-C₆ alkoxy or C₁-C₆ haloalkyl; and
   n is an integer selected from 0-2,
   comprising the steps of:
a. preparing a compound of Formula 5 from a compound of Formula 6 in the presence of a suitable reagent and a suitable solvent (A);

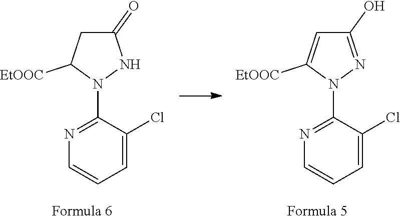 [SMILES: CCOC(=O)C1CC(=O)NN1c1ncccc1Cl ; CCOC(=O)c1cc(O)nn1-c1ncccc1Cl]

b. reacting the compound of Formula 5 with a compound of Formula 10 in the presence of a suitable base (I), a suitable solvent (B), and an optional phase transfer catalyst, to obtain a compound of Formula 4, wherein the compound of Formula 4 is optionally isolated;

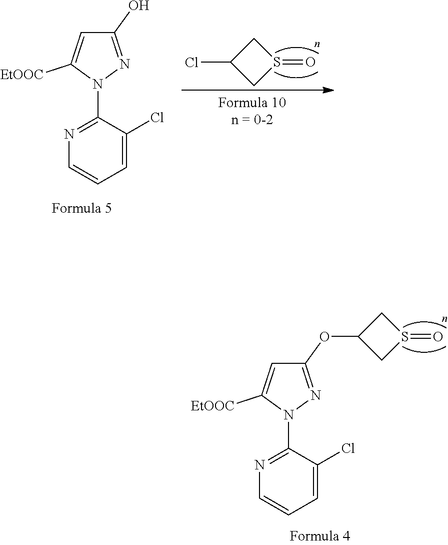

c. hydrolyzing the compound of Formula 4 in the presence of a hydrolyzing agent to obtain a compound of Formula 3; and

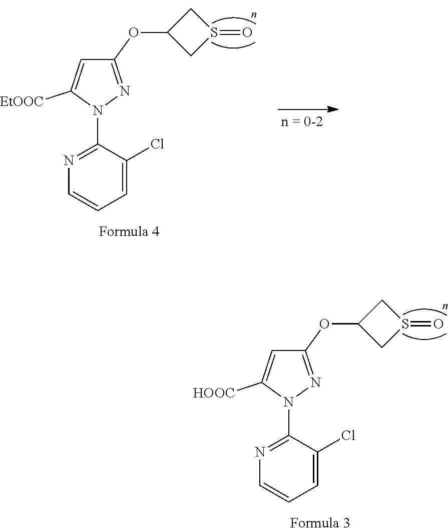

d. reacting the compound of Formula 3 with an acid chloride in a suitable solvent (C) and a suitable base (II) to obtain the anthranilic diamide of Formula 1, its intermediates, N-oxides or salts thereof.

The present invention also provides a method for preparing anthranilic diamide compound of Formula 1, its intermediates, N-oxides or salts thereof,

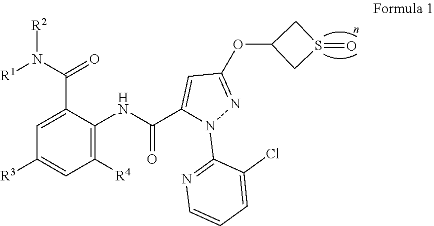

wherein,
R¹ is selected from hydrogen or C₁-C₆ alkyl;
R² is selected from C₁-C₆ alkyl or C₃-C₆ cycloalkyl, wherein said C₁-C₆ alkyl and C₃-C₆ cycloalkyl is optionally substituted with one or more substituents selected from a group consisting of halogen and C₃-C₆ cycloalkyl;
R³ is selected from hydrogen, halogen or cyano;
R⁴ is selected from halogen, C₁-C₆ alkyl, C₁-C₆ alkoxy or C₁-C₆ haloalkyl; and
n is an integer selected from 0-2, comprising the steps of:
a. preparing a compound of Formula 5 from a compound of Formula 6 in the presence of a suitable reagent and a suitable solvent (A);

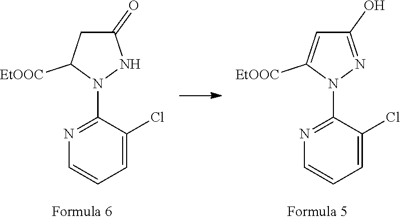 [SMILES: CCOC(=O)C1CC(=O)NN1c1ncccc1Cl ; CCOC(=O)c1cc(O)nn1-c1ncccc1Cl]

b. reacting the compound of Formula 5 with a compound of Formula 10 in the presence of a suitable base (I), a suitable solvent (B), and an optional phase transfer catalyst to obtain a compound of Formula 4, wherein the compound of Formula 4 is optionally isolated;

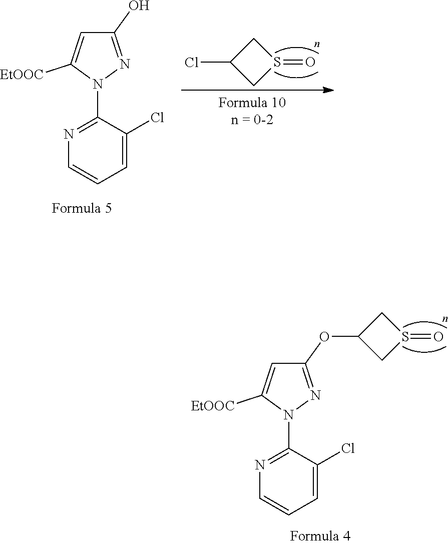

c. hydrolyzing the compound of Formula 4 in the presence of a hydrolyzing agent to obtain a compound of Formula 3; and

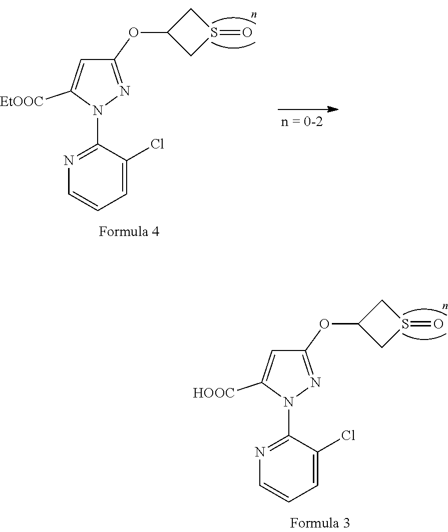

d. (i). reacting the compound of Formula 3 with an acid chloride in a suitable solvent (C) to form an acid chloride of Formula 3A, followed by coupling with a compound of Formula 9 using a suitable base (II) and a suitable solvent (D), to obtain the anthranilic diamide of Formula 1, its intermediates, N-oxides or salts thereof.

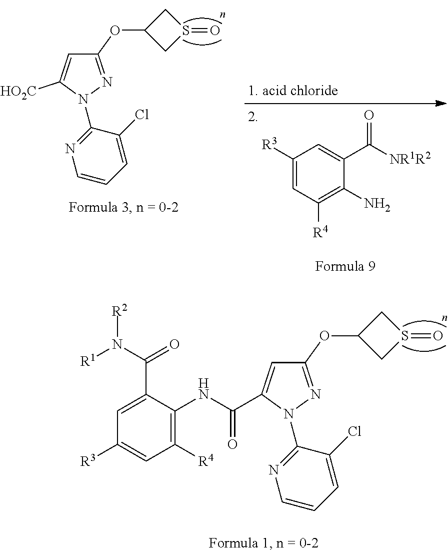

The present invention also provides a method for preparing anthranilic diamide compound of Formula 1, its intermediates, N-oxides or salts thereof,

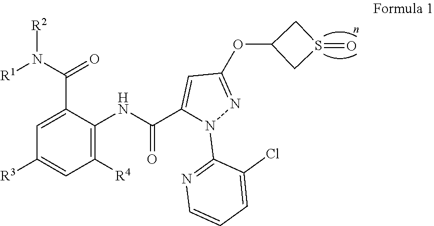

wherein,
R¹ is selected from hydrogen or C₁-C₆ alkyl;
R² is selected from C₁-C₆ alkyl or C₃-C₆ cycloalkyl, wherein said C₁-C₆ alkyl and C₃-C₆ cycloalkyl is optionally substituted with one or more substituents selected from a group consisting of halogen and C₃-C₆ cycloalkyl;
R³ is selected from hydrogen, halogen or cyano;
R⁴ is selected from halogen, C₁-C₆ alkyl, C₁-C₆ alkoxy or C₁-C₆ haloalkyl; and
n is an integer selected from 0-2, comprising the steps of:
   a. preparing a compound of Formula 5 from a compound of Formula 6 in the presence of a suitable reagent and a suitable solvent (A);

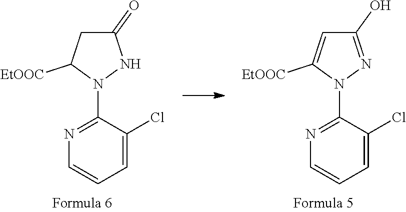 [SMILES: CCOC(=O)C1CC(=O)NN1c1ncccc1Cl ; CCOC(=O)c1cc(O)nn1-c1ncccc1Cl]

b. reacting the compound of Formula 5 with a compound of Formula 10 in the presence of a suitable base (I), a suitable solvent (B), and an optional phase transfer catalyst, to obtain a compound of Formula 4, wherein the compound of Formula 4 is optionally isolated;

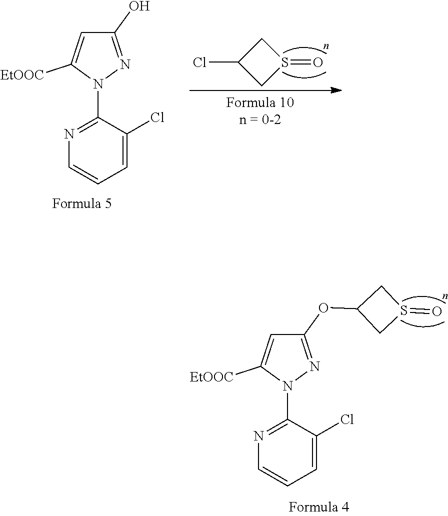

c. hydrolyzing the compound of Formula 4 in the presence of a hydrolyzing agent to obtain a compound of Formula 3;

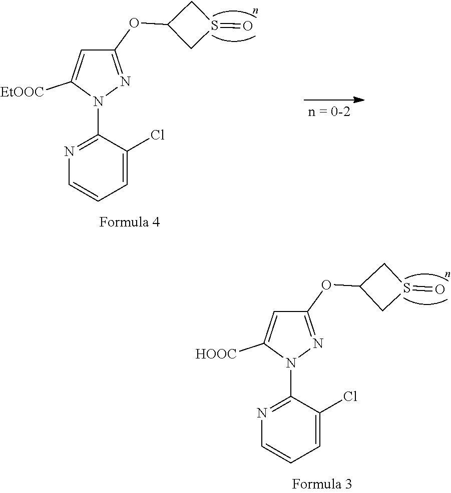

d. (ii). reacting the compound of Formula 3 with a compound of Formula 7 in the presence of a suitable base (II) and an acid chloride to form a compound of Formula 2, wherein the compound of Formula 2 is optionally isolated; and ring opening of the compound of Formula 2 with a compound of Formula 8 in the presence of a suitable base (III) in a suitable solvent (C) to obtain anthranilic diamide of Formula 1, its intermediates, N-oxides or salts thereof,

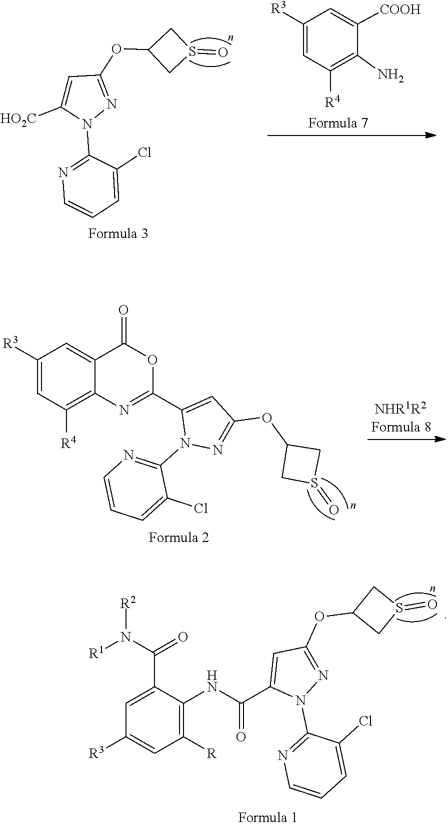

The present invention also provides a method for preparing anthranilic diamide compound of Formula 1, its intermediates, N-oxides or salts thereof,

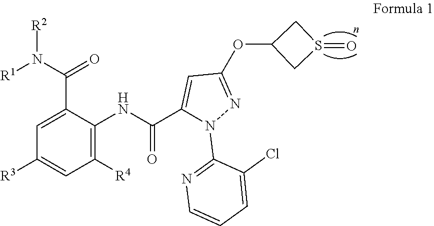

wherein,
   R¹ is selected from hydrogen or C₁-C₆ alkyl;
   R² is selected from C₁-C₆ alkyl or C₃-C₆ cycloalkyl, wherein said C₁-C₆ alkyl and C₃-C₆ cycloalkyl is optionally substituted with one or more substituents selected from a group consisting of halogen and C₃-C₆ cycloalkyl;
   R³ is selected from hydrogen, halogen or cyano;
   R⁴ is selected from halogen, C₁-C₆ alkyl, C₁-C₆ alkoxy or C₁-C₆ haloalkyl; and
   n is an integer selected from 0-2, comprising the steps of:
   a. preparing a compound of Formula 5 from a compound of Formula 6 in the presence of a suitable reagent and a suitable solvent (A);

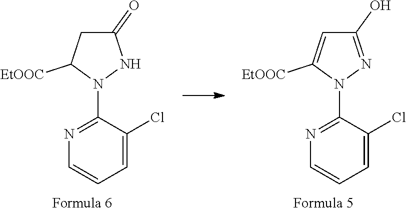 [SMILES: CCOC(=O)C1CC(=O)NN1c1ncccc1Cl ; CCOC(=O)c1cc(O)nn1-c1ncccc1Cl]

b. reacting the compound of Formula 5 with a compound of Formula 10 in the presence of a suitable base (I), a suitable solvent (B), and an optional phase transfer catalyst, to obtain a compound of Formula 4, wherein the compound of Formula 4 is optionally isolated;

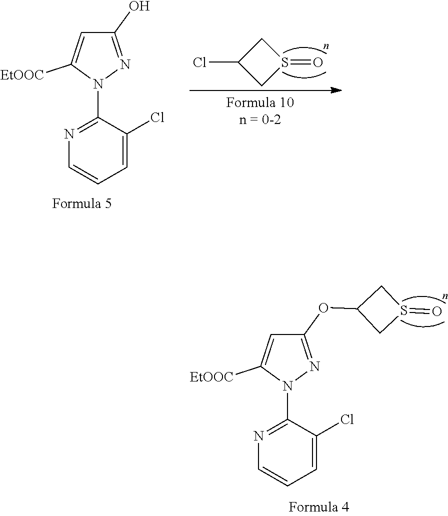

c. hydrolyzing the compound of Formula 4 in the presence of a hydrolyzing agent to obtain a compound of Formula 3; and

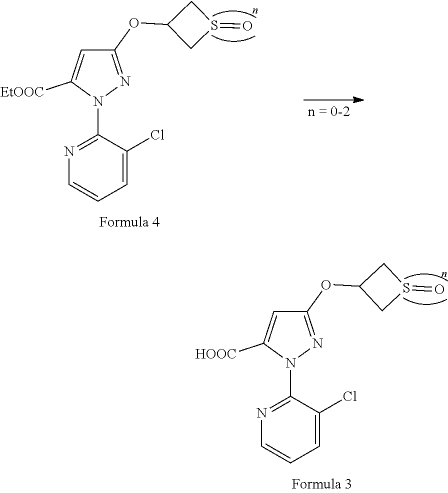

d. (i) reacting the compound of Formula 3 with an acid chloride in a suitable solvent (C) to form an acid chloride of Formula 3A, followed by coupling with a compound of Formula 9 using a suitable base (II) and a suitable solvent (D), to obtain the anthranilic diamide of Formula 1, its intermediates, N-oxides or salts thereof,

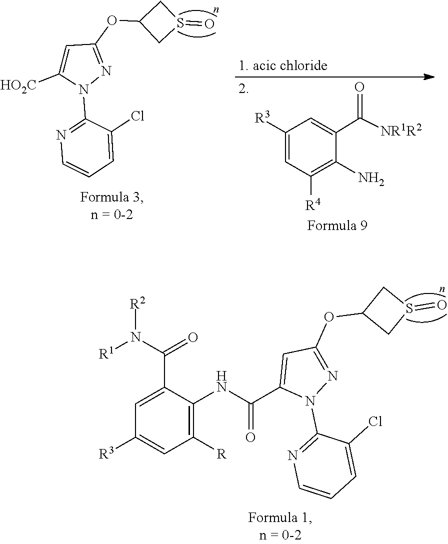

or
d. (ii) reacting the compound of Formula 3 with a compound of Formula 7 in the presence of a suitable base (II) and an acid chloride to form a compound of Formula 2, wherein the compound of Formula 2 is optionally isolated; and ring opening of the compound of Formula 2 with a compound of Formula 8 in the presence of a suitable base (III) in a suitable solvent (C) to obtain anthranilic diamide of Formula 1, its intermediates, N-oxides or salts thereof,

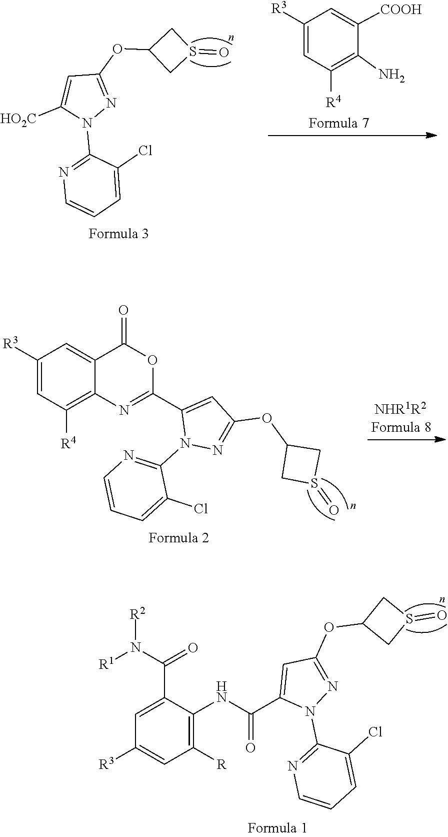

In one embodiment, n=2 for Formula 1, Formula 2, Formula 3, Formula 4 and Formula 10 of the present methods.

In one embodiment, the present invention provides a method for preparing anthranilic diamide compound of Formula 1, wherein R¹ is H or C₁-C₂ alkyl; R² is C₁-C₄ alkyl; R³ is fluoro or chloro; and R⁴ is chloro or C₁-C₂ alkyl.

In a preferred embodiment, the present invention provides a method for preparing anthranilic diamide compound of Formula 1, wherein R¹ is H; R² is iso-propyl, or tert-butyl; R³ is chloro; and R⁴ is methyl.

In one preferred embodiment, the compound of Formula 3 is prepared from the compound of Formula 5 without isolating the compound of Formula 4.

According to one embodiment of the present invention, the conversion of the compound of Formula 5 to the compound of Formula 3 can be carried out in a single step without isolating the compound of Formula 4.

In one embodiment of the present invention, the compound of Formula 3 is converted in-situ to compound of Formula 3A in the presence of a suitable acid chloride and a suitable solvent (C).

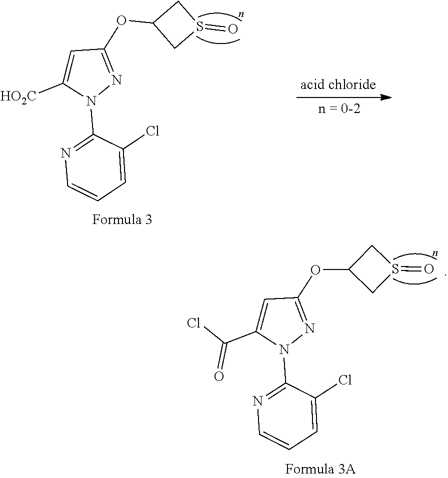

In another embodiment of the present invention, the acid-chloride of Formula 3A in step-d can be isolated before reacting it with the compound of Formula 9.

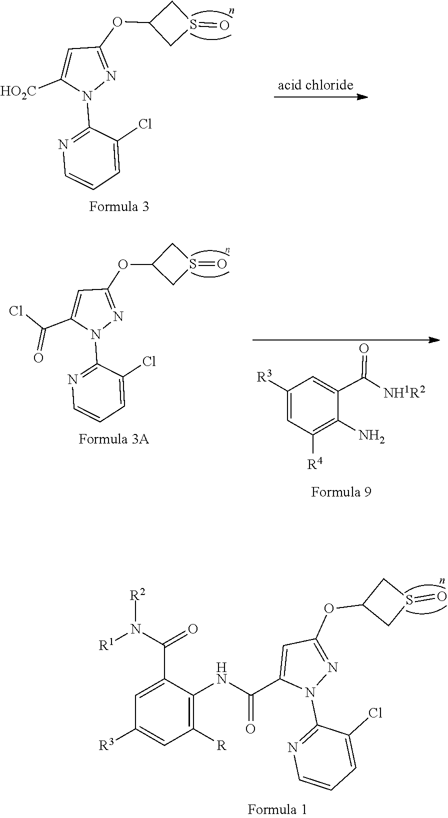

wherein R¹, R², R³, R⁴ and n are as described above.

In one embodiment, the present invention provides the acid chloride of the compound of Formula 3 and is represented as compound of Formula 3A,

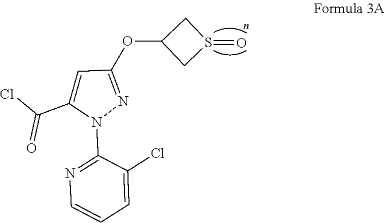

wherein n is an integer selected from 0-2, preferably n is 2.

In another embodiment, the present invention provides a method for preparing a compound of Formula 4, wherein the method comprising step of (step-b):
reacting a compound of Formula 5 with a compound of Formula 10 in the presence of a suitable base (I), a suitable solvent (B), and an optional phase transfer catalyst, to obtain a compound of Formula 4, wherein the compound of Formula 4 is optionally isolated,

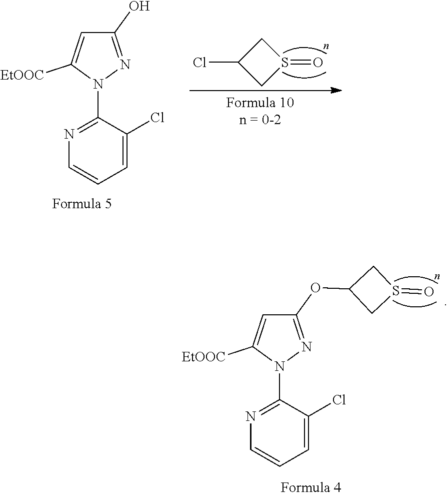

Alternatively, the acid chloride of the compound of Formula 3 can be generated in-situ and then react with a compound of Formula 9 to obtain the anthranilic diamide compound of Formula 1, its intermediates, N-oxides or salts thereof.

The compound of Formula 10 is obtained from a commercial source, or can be synthesized according to known methods as described in PCT application PCT/IN2023/051201 according to the following reaction scheme:

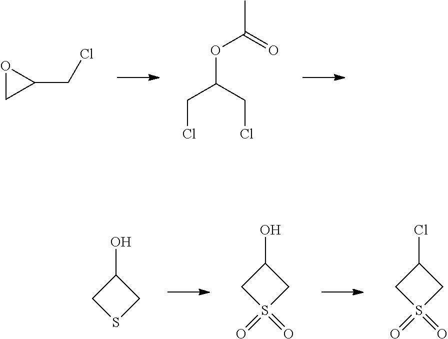

The suitable base selected from base (I), base (II) or base (III), used in the present invention can be independently selected from an organic or inorganic base.

The inorganic base is selected in a non-limiting way from alkali metal hydrogen carbonates, such as lithium hydrogen carbonate (LiHCO₃), sodium hydrogen carbonate (NaHCO₃), potassium hydrogen carbonate (KHCO₃), and cesium hydrogen carbonate (CsHCO₃); alkali/alkaline earth metal carbonates such as sodium carbonate (Na₂CO₃), calcium carbonate (CaCO₃), cesium carbonate (Cs₂CO₃), lithium carbonate (Li₂CO₃), potassium carbonate (K₂CO₃); alkali/alkaline earth metal hydroxides such as lithium hydroxide (LiOH), sodium hydroxide (NaOH), potassium hydroxide (KOH), cesium hydroxide (CsOH), calcium hydroxide (Ca(OH)₂), alkali metal phosphates such as sodium diphosphate (Na₂HPO₄), sodium phosphate (Na₃PO₄), potassium diphosphate (K₂HPO₄), potassium phosphate (K₃PO₄); alkali metal halides such as sodium fluoride (NaF), potassium fluoride (KF), and cesium fluoride (CsF); alkali metal hydrides such as lithium hydride (LiH), sodium hydride (NaH), and potassium hydride (KH); and alkali metal alkoxides such as sodium methoxide (NaOCH₃), sodium ethoxide (NaOCH₂CH₃), sodium tert-butoxide and potassium tert-butoxide and the like.

The organic base is selected in a non-limiting way from amines, such as ethylamine, triethylamine, isopropylamine diisopropylamine, triisopropylamine, pyridine, picoline, piperidine, methylmorpholine, N-methylpiperidine N,N-(dimethylamino)pyridine (DMAP), lutidine, collidine, tetramethylammonium hydroxide, tetrabutylammonium hydroxide, and choline hydroxide; amidines which includes but are not limited to, 1,5,7-triazabicyclo[4.4.0]dec-5-ene (TBD), 2,3,4,6,7,8,9,10-octahydropyrimido-[1,2-a]azepine, 1,8-diazabicyclo[5.4.0]undec-7-ene (DBU), 1,5-diazabicyclo[4.3.0]non-5-ene (DBN), and 1,4-diazabicyclo[2.2.2]octane (DABCO, triethylenediamine).

The suitable solvent selected from solvent (A), solvent (B), solvent (C), or solvent (D), used in the present invention, can be independently selected from aliphatic or aromatic hydrocarbons, halogenated hydrocarbons, ethers, cyclic ethers, nitriles, amides, ketones, acids, alcohols, water, or mixtures thereof. Preferably, the solvent used in the present method can be selected in a non-limiting way from acetonitrile, acetic acid, acetone, hexane, heptane, octane, nonane, decane, dodecane, cycloalkanes such as cyclopropane, cyclobutane, cyclopentane, cyclohexane, cycloheptane, and cyclooctane; N,N-dimethyl formamide, ethylene dichloride, ethyl acetate, toluene, xylene, mesitylene, benzene, halogenated benzene, diisopropyl ether, t-butyl methyl ether, tetrahydrofuran, 2-methyl tetrahydrofuran, dioxane, monoglyme, diglyme, methoxy-methane, methoxy-ethane, ethoxy-ethane, di-methoxyethane, di-ethoxyethane, dichloromethane, chloroform, dichloroethane, N,N-dimethylmethanamide, dimethyl sulfoxide, N-methyl-2-pyrrolidone, 1,3-dimethyl-3,4,5,6-tetrahydro-2(1H)-pyrimidinone, hexamethylphosphoramide, 1,3-dimethyl-2-imidazolidinone, or combinations thereof.

The method for preparing a compound of Formula 3 or Formula 1, wherein the steps of said method step-a, step-b, step-c, and step-d are carried out at a temperature in a range of 0 to 150° C.

The suitable reagent for the step-a of method for preparing a compound of Formula 3 or Formula 1, as disclosed herein, is selected from but is not limited to nitric acid (HNO₃), sulfuric acid, acetic acid, hydrochloric acid, or mixtures thereof. In a preferred embodiment, the suitable reagent used in step-(a) is nitric acid (HNO₃), preferably 70% HNO₃.

In a preferred embodiment, the suitable solvent (A) in step-(a) of method for preparing a compound of Formula 3 or Formula 1 as disclosed herein, is selected from dichloromethane, dichloroethane or acetonitrile, preferably dichloroethane.

In a preferred embodiment, the step-(a) of method for preparing a compound of Formula 3 or Formula 1 as disclosed herein, is usually carried out at a temperature in a range of 0 to 100° C., preferably between 0 to 40° C.

In another preferred embodiment, the step-a of method for preparing a compound of Formula 3 or Formula 1, as disclosed herein, wherein
i. the suitable reagent in the method step-a is selected from nitric acid (HNO₃), sulfuric acid, acetic acid, hydrochloric acid, or mixtures thereof;
ii. the suitable solvent (A) in the method step-a is selected from dichloromethane, dichloroethane or acetonitrile;
iii, the method step-a is carried out at a temperature in a range of 0 to 100° C.

In a preferred embodiment, the suitable base (I) in step-(b) of method for preparing a compound of Formula 3 or Formula 1 as disclosed herein, is selected from but is not limited to alkali or alkaline earth metal carbonates or bicarbonates, alkali/alkaline earth metal hydroxides, alkali metal phosphates, alkali metal alkoxides or alkali metal hydrides; preferably K₂CO₃, Na₂CO₃, NaOH, KOH or K₃PO₄; more preferably K₂CO₃, KOH or K₃PO₄.

In a preferred embodiment, the suitable solvent (B) for step-(b) of method for preparing a compound of Formula 3 or Formula 1 as disclosed herein, is selected from but is not limited to dichloroethane, toluene, xylenes, chlorinated benzene, acetonitrile and dioxane, dimethoxy ethane; preferably mono-chlorobenzene/dichlorobenzene/chlorobenzene.

In a preferred embodiment, the phase transfer catalyst if used in step-(b) of the present methods, is selected from tetraalkylammonium halide, preferably tetrabutylammonium bromide.

In a preferred embodiment, the suitable temperature for step-(b) of method for preparing a compound of Formula 3 or Formula 1 as disclosed herein, is in the range of 25 to 130° C., preferably between 25 to 105° C.

In another preferred embodiment, the step-b of method for preparing a compound of Formula 3 or Formula 1, as disclosed herein, wherein
i. the suitable base (I) in the method step-b is selected from K₂CO₃, Na₂CO₃, NaOH, KOH or K₃PO₄;
ii. the suitable solvent (B) in the method step-b is selected from dichloroethane, toluene, xylenes, chlorinated benzene, acetonitrile dimethoxy ethane and dioxane;
iii, the optional phase transfer catalyst is selected from tetraalkylammonium halide;
iv. the method step-b is carried out at a temperature in a range of 25 to 130° C.

The suitable hydrolyzing agent used in step-(c) of method for preparing a compound of Formula 3 or Formula 1 as disclosed herein, is an acid. In a preferred embodiment, the acid used for the ester hydrolysis in step-(c) is selected from but is not limited to aqueous sulfuric acid (aqueous H₂SO₄) and hydrochloric acid (HCl). In a more preferred embodiment, the hydrolyzing agent used in step-(c) of the present invention is 10-50% aqueous sulfuric acid; preferably 20% aqueous H₂SO₄.

The suitable solvent for the hydrolysis in step-(c) of method for preparing a compound of Formula 3 or Formula 1 as disclosed herein, is selected from but is not limited to acetic acid (AcOH), water or acetonitrile; preferably acetic acid (AcOH).

In a preferred embodiment, the suitable temperature for step-(c) of method for preparing a compound of Formula 3 or Formula 1 as disclosed herein, is in the range of 50 to 130° C., preferably between 70 to 105° C.

In another preferred embodiment, the step-c of method for preparing a compound of Formula 3 or Formula 1, as disclosed herein, wherein
i. the hydrolyzing agent in the method step-c is selected from an acid;
ii. the suitable solvent in the method step-c is selected from acetic acid (AcOH), water or acetonitrile;
iii, the method step-c is performed within a temperature range of 50 to 130° C.

The acid chloride used in step-(d), step-(d(i)) or step-(d(ii)) of method for preparing a compound of Formula 1 as disclosed herein, is selected from but is not limited to thionyl chloride (SOCl₂), mesityl chloride (MsCl, methanesulfonyl chloride), phosphorus pentachloride (PCl₅), phosphorus trichloride (PCl₃), oxalyl chloride, triphosgene and phosgene. In a preferred embodiment, the acid chloride is selected from thionyl chloride (SOCl₂), mesityl chloride (MsCl) or phosgene (COCl₂).

In a preferred embodiment, the base (II) and the base (III) used in step-(d), step-(d(i)) or step-(d(ii)) of method for preparing a compound of Formula 1 as disclosed herein, is independently selected from but is not limited to an inorganic base such as alkali or alkaline earth metal carbonate, bicarbonate, hydroxide, hydrides or alkoxide; organic base such as isopropyl amine, triethylamine, diisopropyl ethyl amine, triisopropyl amine, pyridine, picoline, N-methylmorpholine, N-methylpiperidine, N,N-(dimethylamino)pyridine (DMAP), lutidine, collidine, tetramethylammonium hydroxide, tetrabutylammonium hydroxide; amidines such as, 1,8-diazabicyclo[5.4.0]undec-7-ene (DBU), 1,4-diazabicyclo[2.2.2]octane (DABCO), 1,5,7-triazabicyclo[4.4.0]dec-5-ene (TBD), or 1,5-diazabicyclo[4.3.0]non-5-ene (DBN). In a more preferred embodiment, the base (II) and the base (III) are independently selected from triethylamine, isopropyl amine, pyridine, picoline or mixtures thereof.

In a preferred embodiment, the suitable solvent (C) and the solvent (D) in step-(d), step-(d(i)) or step-(d(ii)) of method for preparing a compound of Formula 1 as disclosed herein, are independently selected from dichloroethane, acetonitrile (ACN), N,N-dimethylformamide (DMF), ethyl acetate, pyridine, picoline, or mixtures thereof.

The reaction temperature for performing step-(d), step-(d(i)) and step-(d(ii)) of method for preparing a compound of Formula 1 as disclosed herein, is in the range of 0 to 80° C.

In another preferred embodiment, the step-d, step-(d(i)), or step-(d(ii)) of method for preparing a compound of Formula 1, as disclosed herein, wherein
i. the acid chloride in the method step-d, step-(d(i)) or step-(d(ii)) is selected from thionyl chloride (SOCl₂), mesityl chloride (MsCl), phosphorus pentachloride (PCl₅), phosphorus trichloride (PCl₃), oxalyl chloride, triphosgene or phosgene;
ii. the suitable solvent (C) and solvent (D) in the method step-d, step-(d(i)) or step-(d(ii)) are independently selected from dichloroethane, acetonitrile (ACN), N,N-dimethylformamide (DMF), ethyl acetate, pyridine, picoline, or mixtures thereof;
iii, the suitable base (II) and base (III) in the method step-d, step-(d(i)) or step-(d(ii)), are independently selected from alkali or alkaline earth metal carbonate, bicarbonate, hydroxide, hydrides or alkoxide, isopropyl amine, triethylamine, diisopropyl ethyl amine, triisopropyl amine, pyridine, picoline, N-methylmorpholine, N-methylpiperidine, N,N-(dimethylamino)pyridine (DMAP), lutidine, collidine, tetramethylammonium hydroxide, tetrabutylammonium hydroxide, 1,8-diazabicyclo[5.4.0]undec-7-ene (DBU), 1,4-diazabicyclo[2.2.2]octane (DABCO), 1,5,7-triazabicyclo[4.4.0]dec-5-ene (TBD), or 1,5-diazabicyclo[4.3.0]non-5-ene (DBN);
iv. the method step-d, step-(d(i)) or step-(d(ii)) is performed at a temperature within a range of 0 to 80° C.

The reaction time is not critical and depends on the batch size, temperature, type of reaction, solvent and the used reagents and is usually between few minutes to few hours.

The methods disclosed in the present invention for preparing the anthranilic diamides of Formula 1, the intermediates of Formula 3, Formula 4 or their respective N-oxides or salts as described above, employ easy conversions, readily available starting materials, reagents and reaction conditions, which make them amenable for commercial scale applications. Further, the methods disclosed in the present invention provide the required intermediate and the final compound in high yield and chemical purity.

A person skilled in the art knows the best work-up procedures of the reaction mixtures after the end of the respective reactions. The work-up is usually carried out by isolation of the product, and optionally washing with solvents, and further optionally drying of the product if useful or required.

According to the methods disclosed in the present invention, the products obtained in individual steps may be isolated, or alternatively is not isolated from the reaction mixture and directly used for the subsequent reaction step. The isolation of the reaction products can be carried out by a technique which includes but is not limited to decantation, filtration, centrifugation, evaporation, liquid-liquid extraction, distillation, recrystallization, chromatography and the like, or a combination thereof.

The reaction steps according to the invention are generally carried out under atmospheric pressure. Alternatively, however, it is also possible to carry out the reaction steps of the present method under reduced pressure or higher pressure.

The invention is further illustrated by the following examples which are provided to be exemplary of the invention, and do not limit the scope of the invention. While the present invention has been described in terms of its specific embodiments, certain modifications and equivalents will be apparent to those skilled in the art and are intended to be included within the scope of the present invention.

The present invention provides a method for preparing anthranilic diamides of Formula 1, the intermediates of Formula 3, Formula 4 or their respective N-oxides or salts as described above, wherein the steps can be carried out in a batch, semi-continuous or continuous reaction mode, specifically also under semi-continuous flow or continuous flow reaction conditions.

Although the subject matter has been described in considerable detail with reference to certain examples and implementations thereof, other implementations are possible.

### EXAMPLES

The disclosure will now be illustrated with the working examples, which is intended to illustrate the working of disclosure and not intended to take restrictively to imply any limitations on the scope of the present disclosure. Unless defined otherwise, all technical and scientific terms used herein have the same meaning as commonly understood to one ordinary person skilled in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may apply.

### Experimental Examples

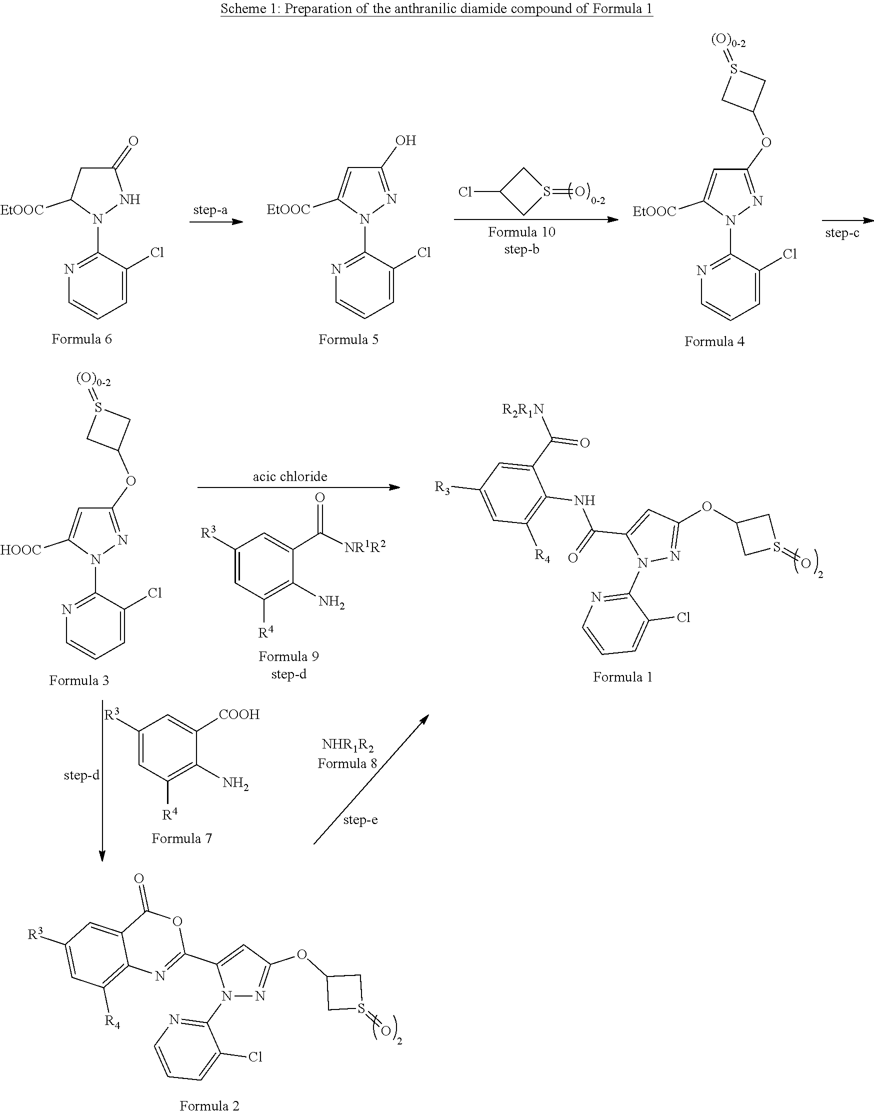

All the solvents and the reagents used in the present invention are obtained from commercial sources. Reactants such as ethyl 2-(3-chloro-2-pyridinyl)-5-oxo-3-pyrazolidinecarboxylate (compound of Formula 6), anthranilic acid based reactants (compounds of Formula 7) and amines of Formula 8 were obtained either from commercial suppliers or prepared according to known literature procedure. The amine of Formula 9 was prepared according to the literature procedure or as described in the prior art e.g. WO 2020170092 and WO 2022064454.

### Example 1: Preparation of the Anthranilic Diamide Compound of Formula 1

### Step a: Synthesis of ethyl 1-(3-chloropyridin-2-yl)-3-hydroxy-1H-pyrazole-5-carboxylate

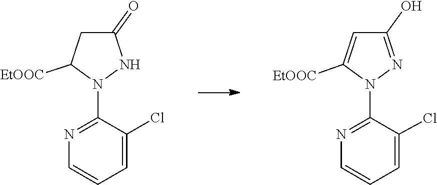 [SMILES: CCOC(=O)C1CC(=O)NN1c1ncccc1Cl ; CCOC(=O)c1cc(O)nn1-c1ncccc1Cl]

To a stirred suspension of ethyl 2-(3-chloropyridin-2-yl)-5-oxopyrazolidine-3-carboxylate (compound of Formula 6, 100 g, 371 mmol) in dichloroethane (500 mL), 70% aqueous nitric acid (16.7 g, 185 mmol) was added at 0-5° C. The temperature of the reaction mass was raised to 20-30° C., and stirring was continued. After completion of the reaction, the reaction mass was cooled to 0-5° C. The solid obtained was filtered, washed with water (500 mL) and dried to afford ethyl 1-(3-chloropyridin-2-yl)-3-hydroxy-1H-pyrazole-5-carboxylate (Formula 5) (68 g, 68% yield).

¹H NMR (DMSO-d₆, 400 MHz): δ 8.12-8.10 (dd, J=1.6 Hz, 1H), 7.84-7.82 (dd, J=1.6 Hz, 1H), 6.98-6.95 (dd, J=4.8 Hz, 1H), 5.28-5.23 (m, 1H), 4.11 (q, 2H), 3.67 (s, 3H), 3.54-3.47 (.m, 1H), 3.20-3.16 (m, 1H), 1.14 (t, 3H), 3.31 (m, 1H), 2.93 (m, 1H), 1.15 (t, 3H) MS: m/z=348.0 [M+H]⁺.

### Step-b: Synthesis of ethyl 1-(3-chloropyridin-2-yl)-3-((1,1-dioxidothietan-3-yl)oxy)-1H-pyrazole-5-carboxylate

### Method 1: Reaction Using Potassium Carbonate as Base

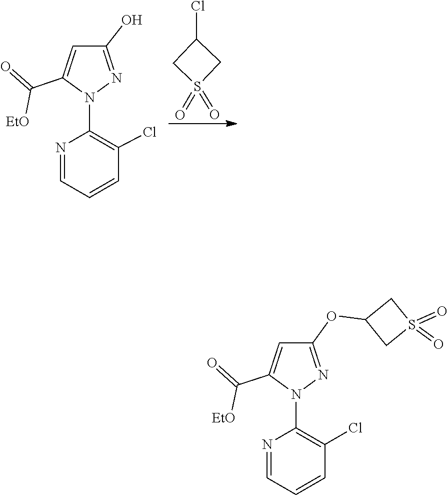

To a stirred suspension of ethyl 1-(3-chloropyridin-2-yl)-3-hydroxy-1H-pyrazole-5-carboxylate (86 g, 300 mmol) in chlorobenzene (172 mL), potassium carbonate (2.0 eq., 85 g, 600 mmol) was added at 25-30° C. The temperature of the reaction mass was raised to 70-75° C., and stirring was continued further for 45 minutes. To this reaction mass, a solution of 3-chlorothietane 1,1-dioxide (Formula 10) (2.0 eq., 89 g, 600 mmol) in chlorobenzene (172 mL) was added at 70-75° C. The temperature of the reaction mass was raised to 80-85° C., and stirring was continued further for 3 h. After completion of the reaction, the reaction mass was cooled to 60-65° C. and filtered. The filtrate was concentrated under reduced pressure to afford ethyl 1-(3-chloropyridin-2-yl)-3-((1,1-dioxidothietan-3-yl)oxy)-1H-pyrazole-5-carboxylate (Formula 4) (135.7 g) as a crude product which was further used for step-c.

HPLC purity (%)—86.6; MS-m/z 371 [M+H]⁺.

### Method 2: Reaction Using Tribasic Potassium Phosphate as Base

To a stirred suspension of ethyl 2-(3-chloropyridin-2-yl)-5-oxopyrazolidine-3-carboxylate (5 g, 18.7 mmol) in chlorobenzene (50 mL), 3-chlorothietane 1,1-dioxide (15.01 g, 37.4 mmol), tetrabutyl ammonium bromide (1.5 g, 4.7 mmol) and tribasic potassium phosphate (13.9 g, 65.4 mmol) were added at 25-30° C. The temperature of the reaction mass was raised to 80-85° C., and stirring was continued for 30 h. After completion of the reaction, the reaction mass was cooled to 60-65° C. and filtered. The filtrate was concentrated under reduced pressure to afford ethyl 1-(3-chloropyridin-2-yl)-3-((1,1-dioxidothietan-3-yl)oxy)-1H-pyrazole-5-carboxylate (Formula 4) (11.9 g, 77% yield).

### Method 3: Reaction Using Potassium Hydroxide as Base

To a stirred suspension of ethyl 2-(3-chloropyridin-2-yl)-5-oxopyrazolidine-3-carboxylate (0.5 g, 1.868 mmol) in chlorobenzene (5 mL), 3-chlorothietane 1,1-dioxide (0.52 g, 3.74 mmol), tetrabutyl ammonium bromide (0.15 g, 0.47 mmol) and potassium hydroxide (0.26 g, 4.67 mmol) were added at 25-30° C. The temperature of the reaction mass was raised to 80-85° C., and stirring was continued for 24 h. After completion of the reaction, the reaction mass was cooled to 60-65° C. and filtered. The filtrate was concentrated under reduced pressure to afford ethyl 1-(3-chloropyridin-2-yl)-3-((1,1-dioxidothietan-3-yl)oxy)-1H-pyrazole-5-carboxylate (Formula 4) as a crude product which was used for the next step.

### Step-c: Synthesis of 1-(3-chloropyridin-2-yl)-3-((1,1-dioxidothietan-3-yl)oxy)-1H-pyrazole-5-carboxylic acid (Formula 3)

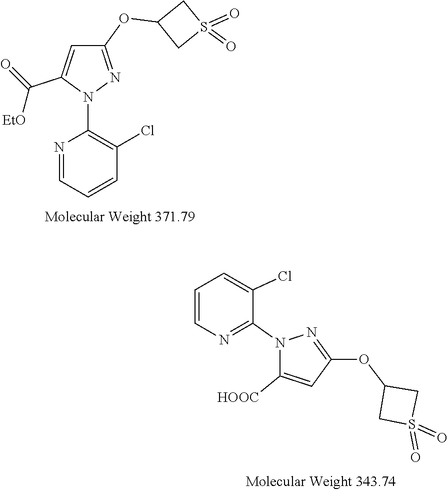 [SMILES: CCOC(=O)c1cc(OC2CS(=O)(=O)C2)nn1-c1ncccc1Cl ; O=C(O)c1cc(OC2CS(=O)(=O)C2)nn1-c1ncccc1Cl]

To a stirred mixture of acetic acid (80 mL) and ethyl 1-(3-chloropyridin-2-yl)-3-((1,1-dioxidothietan-3-yl)oxy)-1H-pyrazole-5-carboxylate (crude product from step-b, method 1, 135.7 g), 20% aqueous sulfuric acid (400 mL) was added. The temperature of the reaction mass was raised to 95-100° C., and stirring was continued further for 24 h. After completion of the reaction, the reaction mass was cooled to 0-5° C. The solid obtained was washed with water (172 mL) and dried to afford 1-(3-chloropyridin-2-yl)-3-((1,1-dioxidothietan-3-yl)oxy)-1H-pyrazole-5-carboxylic acid (72 g, 60% yield) (Formula 3).

¹H-NMR (400 MHz, Chloroform-D) δ 8.48 (dd, J=4.7, 1.7 Hz, 1H), 7.93 (dd, J=8.3, 1.5 Hz, 1H), 7.44 (dd, J=8.1, 4.7 Hz, 1H), 6.58 (s, 1H), 5.38-5.32 (m, 1H), 4.61-4.55 (m, 2H), 4.37-4.31 (m, 2H).

MS: m/z=343.0 [M+H]⁺.

### In-Situ Step-b and Step-c: In-Situ Preparation of 1-(3-chloropyridin-2-yl)-3-((1,1-dioxidothietan-3-yl)oxy)-1H-pyrazole-5-carboxylic acid from ethyl 1-(3-chloropyridin-2-yl)-3-hydroxy-1H-pyrazole-5-carboxylate

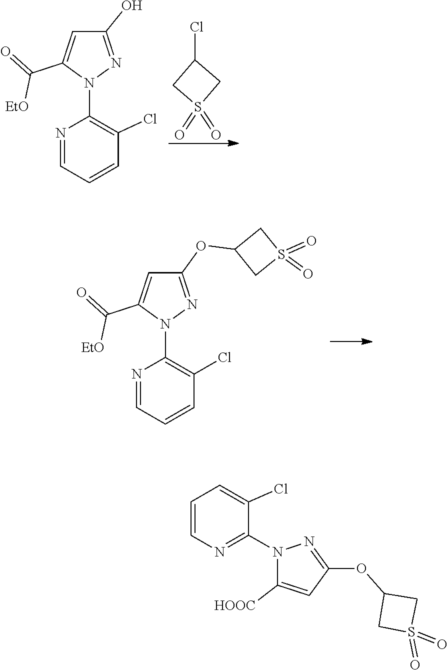

To a stirred suspension of ethyl 1-(3-chloropyridin-2-yl)-3-hydroxy-1H-pyrazole-5-carboxylate, 3-chlorothietane 1,1-dioxide (2.0 eq., 26.3 g, 185 mmol) and potassium carbonate (1.2 eq., 15.65 g, 111 mmol), chlorobenzene (50 mL) was added at 25-30° C. The temperature of the reaction mass was raised to 70-80° C., and stirring was continued for 3 h. After completion of the reaction, the reaction mass was cooled to 25-30° C. Acetic acid (25 mL) and 20% aqueous sulfuric acid (125 mL) were added to the above suspension. The temperature of the reaction mass was raised to 95-100° C., and the mixture was stirred for 24 h. After completion of the reaction, the reaction mass was cooled to 0-5° C., stirred for 1-2 h, and the obtained solid was filtered, washed with water (100 mL) and dried to afford 1-(3-chloropyridin-2-yl)-3-((1,1-dioxidothietan-3-yl)oxy)-1H-pyrazole-5-carboxylic acid (Formula 3) (16.8 g, 48.9 mmol, 90.29% yield).

### Step-d: Synthesis of N-(2-(tert-butylcarbamoyl)-4-chloro-6-methylphenyl)-1-(3-chloropyridin-2-yl)-3-((1,1-dioxidothietan-3-yl)oxy)-1H-pyrazole-5-carboxamide (Formula 1)

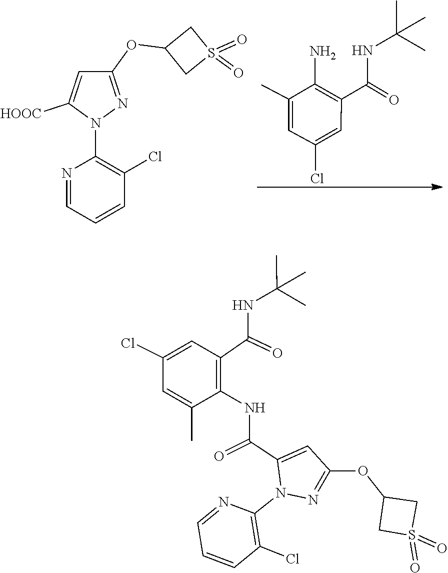

A suspension of 1-(3-chloropyridin-2-yl)-3-((1,1-dioxidothietan-3-yl)oxy)-1H-pyrazole-5-carboxylic acid (1.2 g, 3.09 mmol) and 2-amino-N-(tert-butyl)-5-chloro-3-methylbenzamide (Formula 9) (1.05 eq., 0.8 g, 3.24 mmol) in acetonitrile (6 mL) was cooled to 0-5° C. and stirred for 10-15 minutes. To this suspension, 3-methylpyridine (2.301 g, 2.4 ml, 24.70 mmol) was added dropwise over a period of 5 minutes, and stirring was continued further for 10-15 minutes, followed by the dropwise addition of methanesulfonyl chloride (1.061 g, 0.717 ml, 9.26 mmol) over a period of 5 minutes. The temperature of the reaction mass was raised slowly to 25-30° C., and stirring was continued for further for 5 h. After completion of the reaction, the reaction mass was concentrated under reduced pressure to remove acetonitrile to obtain a residue. Acetone (3 mL) and water (5 mL) were added to the residue and the mixture was stirred at 0-5° C. The solid obtained was filtered, washed with water (5 mL) and dried to afford N-(2-(tert-butylcarbamoyl)-4-chloro-6-methylphenyl)-1-(3-chloropyridin-2-yl)-3-((1,1-dioxidothietan-3-yl)oxy)-1H-pyrazole-5-carboxamide (Formula 1) (1.4 g, 2.472 mmol, 80.04% yield). MS: m/z=566.0 [M+H]⁺.

### Alternative Step-d

### Synthesis of 6-chloro-2-(1-(3-chloropyridin-2-yl)-3-((1,1-dioxidothietan-3-yl)oxy)-1H-pyrazol-5-yl)-8-methyl-4H-benzo[d][1,3]oxazin-4-one

A stirred suspension of 1-(3-chloropyridin-2-yl)-3-((1,1-dioxidothietan-3-yl)oxy)-1H-pyrazole-5-carboxylic acid (Formula 3) (50.0 g, 131 mmol) and 2-amino-5-chloro-3-methylbenzoic acid (Formula 7) (25.6 g, 138 mmol) in acetonitrile (400 mL) was cooled to 0-10° C., and pyridine (83 g, 1052 mmol) was added at 0-10° C. To this reaction mixture, methane sulfonyl chloride (45.2 g, 394 mmol) was added dropwise at 0-10° C. The resulting reaction mixture was stirred for 1-2 h at 25-30° C. After completion of the reaction, the reaction mixture was cooled to 0-5° C. and filtered. The filter cake was washed with water (125 g) and dried under reduced pressure to obtain 6-chloro-2-(1-(3-chloropyridin-2-yl)-3-((1,1-dioxidothietan-3-yl)oxy)-1H-pyrazol-5-yl)-8-methyl-4H-benzo[d][1,3]oxazin-4-one (Formula 2) (63 g, 97% yield).

### Synthesis of N-(2-(tert-butylcarbamoyl)-4-chloro-6-methylphenyl)-1-(3-chloropyridin-2-yl)-3-((1,1-dioxidothietan-3-yl)oxy)-1H-pyrazole-5-carboxamide

To a stirred suspension of 6-chloro-2-(1-(3-chloropyridin-2-yl)-3-((1,1-dioxidothietan-3-yl)oxy)-1H-pyrazol-5-yl)-8-methyl-4H-benzo[d][1,3]oxazin-4-one (43.5 g, 87 mmol) in N,N-dimethylformamide (87 mL), tert-butyl amine (Formula 8) (9.58 g, 131 mmol) was added dropwise over 30-40 minutes at 10-20° C. The reaction mixture was stirred for 6-8 h at 25-30° C. After completion of the reaction, excess tert-butyl amine was distilled off from the reaction mixture under reduced pressure. To the residue, isopropanol (304.5 mL) was added, and the resulting reaction mixture was stirred for 6-8 h at 25-30° C. The solid obtained was filtered, the wet cake obtained was washed with acetone (87 mL) and dried under reduced pressure to obtain N-(2-(tert-butylcarbamoyl)-4-chloro-6-methylphenyl)-1-(3-chloropyridin-2-yl)-3-((1,1-dioxidothietan-3-yl)oxy)-1H-pyrazole-5-carboxamide (Formula 1) (43 g, 86% yield).

1. A method for preparing a compound of Formula 3, its intermediates, N-oxides or salts thereof, 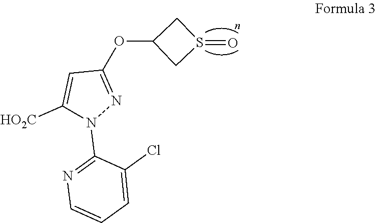
wherein n is an integer selected from 0-2,
comprising the steps of:
a. preparing a compound of Formula 5 from a compound of Formula 6 in the presence of a suitable reagent and a suitable solvent (A), 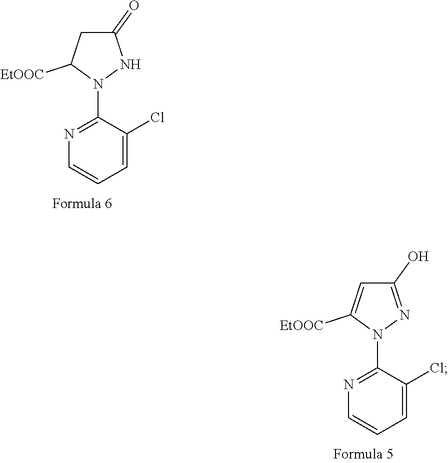 [SMILES: CCOC(=O)C1CC(=O)NN1c1ncccc1Cl ; CCOC(=O)c1cc(O)nn1-c1ncccc1Cl]
b. reacting the compound of Formula 5 with a compound of Formula 10 in the presence of a suitable base (I), a suitable solvent (B), and an optional phase transfer catalyst, to obtain a compound of Formula 4, wherein the compound of Formula 4 is optionally isolated, 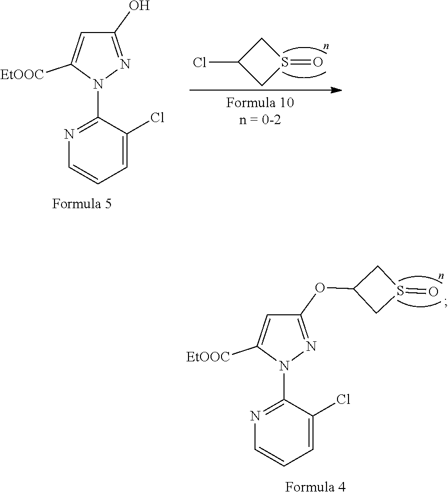
and
c. hydrolyzing the compound of Formula 4 in the presence of a hydrolyzing agent to obtain the compound of Formula 3,

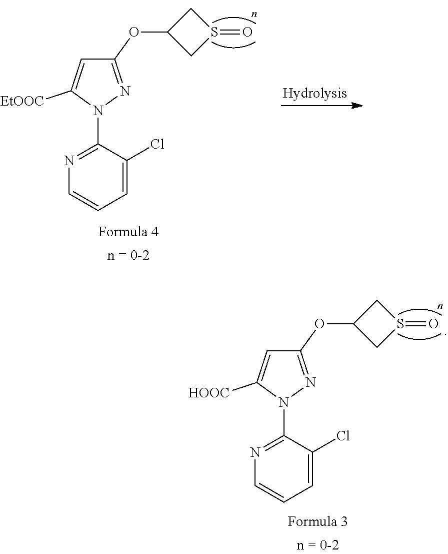

2. The method as claimed in claim 1, wherein said method further comprises preparing anthranilic diamide of Formula 1, its intermediates, N-oxides or salts thereof, 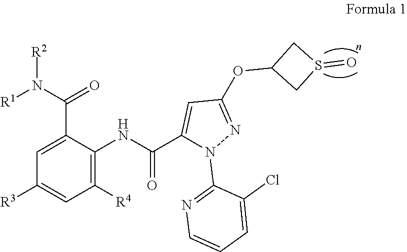
wherein,
R¹ is selected from hydrogen or C₁-C₆ alkyl;
R² is selected from C₁-C₆ alkyl or C₃-C₆ cycloalkyl, wherein said C₁-C₆ alkyl and C₃-C₆ cycloalkyl is optionally substituted with one or more substituents selected from a group consisting of halogen and C₃-C₆ cycloalkyl;
R³ is selected from hydrogen, halogen or cyano;
R⁴ is selected from halogen, C₁-C₆ alkyl, C₁-C₆ alkoxy or C₁-C₆ haloalkyl; and
n is an integer selected from 0-2,
comprising the steps of:
a. preparing a compound of Formula 5 from a compound of Formula 6 in the presence of a suitable reagent and a suitable solvent (A); 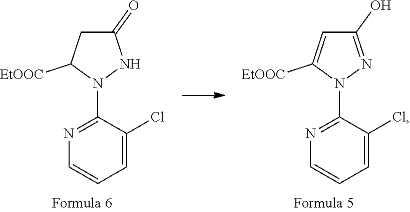 [SMILES: CCOC(=O)C1CC(=O)NN1c1ncccc1Cl ; CCOC(=O)c1cc(O)nn1-c1ncccc1Cl]
b. reacting the compound of Formula 5 with a compound of Formula 10 in the presence of a suitable base (I), a suitable solvent (B), and an optional phase transfer catalyst, to obtain a compound of Formula 4, wherein the compound of Formula 4 is optionally isolated, 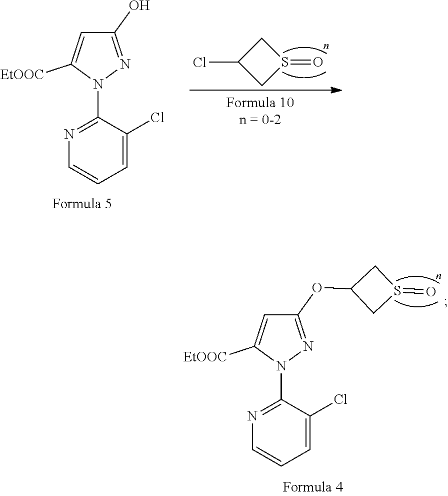
c. hydrolyzing the compound of Formula 4 in the presence of a hydrolyzing agent to obtain a compound of Formula 3, 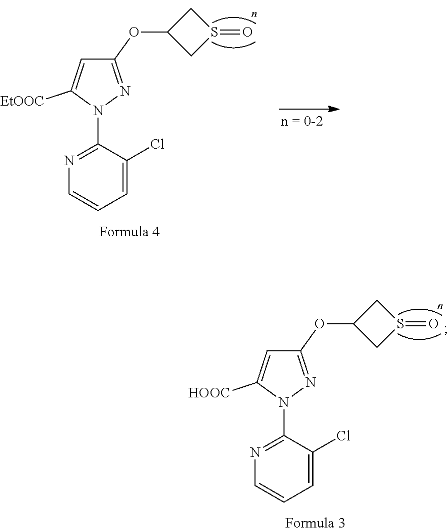
and
d. reacting the compound of Formula 3 with an acid chloride in a suitable solvent (C) and a suitable base (II) to obtain the anthranilic diamide of Formula 1, its intermediates, N-oxides or salts thereof.

3. The method as claimed in claim 2, wherein in step-d, the compound of Formula 3 is reacted with an acid chloride in a suitable solvent (C) to form an acid chloride of Formula 3A, followed by coupling with a compound of Formula 9 using a suitable base (II) and a suitable solvent (D), to obtain the anthranilic diamide of Formula 1, its intermediates, N-oxides or salts thereof. 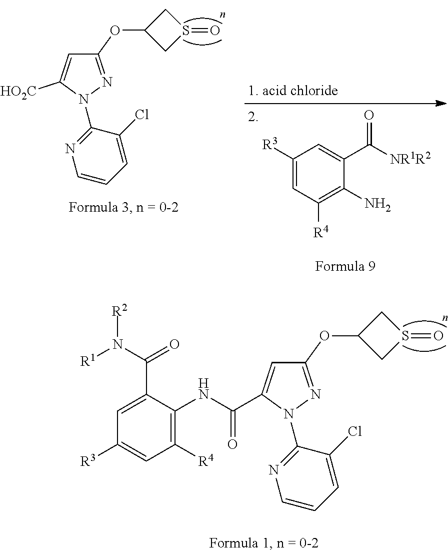
wherein R¹, R², R³, R⁴ and n are as defined in claim 2.

4. The method as claimed in claim 2, wherein in step-d, the compound of Formula 3 is reacted with a compound of Formula 7 in the presence of a suitable base (II) and an acid chloride to form a compound of Formula 2, wherein the compound of Formula 2 is optionally isolated; and ring opening of the compound of Formula 2 with a compound of Formula 8 in the presence of a suitable base (III) in a suitable solvent (C) to obtain anthranilic diamide of Formula 1, its intermediates, N-oxides or salts thereof, 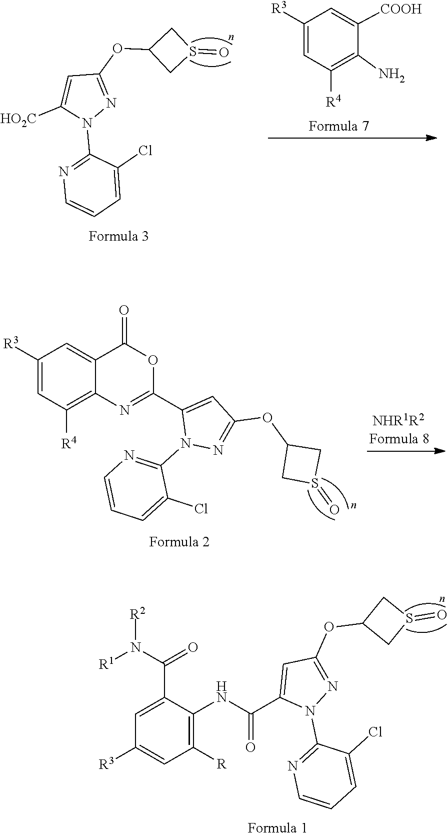
wherein R¹, R², R³, R⁴ and n are as defined in claim 2.

5. The method as claimed in claim 2, wherein R¹ is H or C₁-C₂ alkyl; R² is C₁-C₄ alkyl; R³ is fluoro or chloro; and R⁴ is chloro or C₁-C₂ alkyl.

6. The method as claimed in claim 5, wherein R¹ is H; R² is isopropyl or tert-butyl; R³ is chloro; and R⁴ is methyl.

7. The method as claimed in claim 1, wherein the compound of Formula 3 is prepared from the compound of Formula 5 in a single step without isolating the compound of Formula 4.

8. The method as claimed in claim 2, wherein in step-d, the compound of Formula 3 is converted in-situ to a compound of Formula 3A in the presence of a suitable acid chloride and a suitable solvent (C).

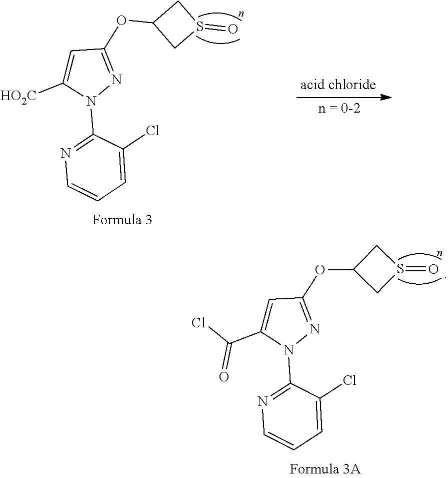

9. The method as claimed in claim 2, wherein in step-d, the compound of formula 3 is converted to a compound of Formula 3A in the presence of a suitable acid chloride and a suitable solvent (C), wherein the acid-chloride of Formula 3A can be isolated.

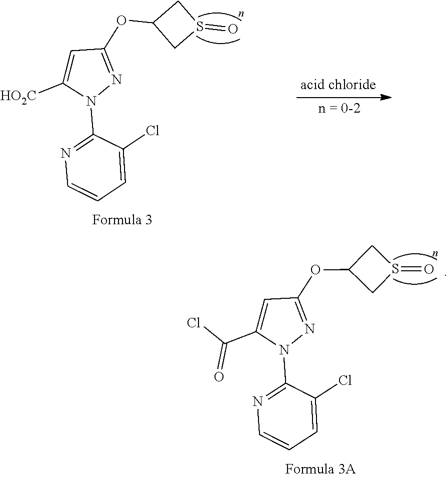

10. A method for preparing a compound of Formula 4, wherein the method comprising step of (step b):
reacting a compound of Formula 5 with a compound of Formula 10 in the presence of a suitable base (I), a suitable solvent (B), and an optional phase transfer catalyst, to obtain a compound of Formula 4, wherein the compound of Formula 4 is optionally isolated.

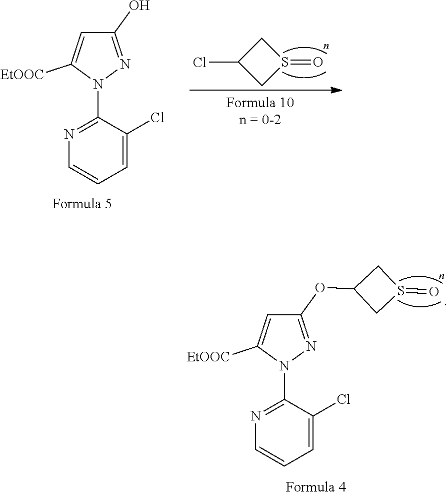

11. The method as claimed in claim 1, wherein n=2.

12. The method as claimed in claim 1, wherein the suitable base (I), base (II) or base (III) are independently selected from an inorganic or organic base.

13. The method as claimed in claim 12, wherein the inorganic base can be selected from lithium hydrogen carbonate (LiHCO₃), sodium hydrogen carbonate (NaHCO₃), potassium hydrogen carbonate (KHCO₃), cesium hydrogen carbonate (CsHCO₃), sodium carbonate (Na₂CO₃), calcium carbonate (CaCO₃), cesium carbonate (Cs₂CO₃), lithium carbonate (Li₂CO₃), potassium carbonate (K₂CO₃), lithium hydroxide (LiOH), sodium hydroxide (NaOH), potassium hydroxide (KOH), cesium hydroxide (CsOH), calcium hydroxide (Ca(OH)₂), sodium diphosphate (Na₂HPO₄), sodium phosphate (Na₃PO₄), potassium diphosphate (K₂HPO₄), potassium phosphate (K₃PO₄), sodium fluoride (NaF), potassium fluoride (KF), cesium fluoride (CsF), lithium hydride (LiH), sodium hydride (NaH), potassium hydride (KH), sodium methoxide (NaOCH₃), sodium ethoxide (NaOCH₂CH₃), sodium tert-butoxide or potassium tert-butoxide.

14. The method as claimed in claim 12, wherein the organic base can be selected from ethylamine, triethylamine, isopropylamine, diisopropylamine, triisopropylamine, pyridine, picoline, piperidine, methylmorpholine, N-methylpiperidine, N,N-(dimethylamino)pyridine (DMAP), lutidine, collidine, tetramethylammonium hydroxide, tetrabutylammonium hydroxide, choline hydroxide, 1,5,7-triazabicyclo[4.4.0]dec-5-ene (TBD), 2,3,4,6,7,8,9,10-octahydropyrimido-[1,2-a]azepine, 1,8-diazabicyclo[5.4.0]undec-7-ene (DBU), 1,5-diazabicyclo[4.3.0]non-5-ene (DBN), or 1,4-diazabicyclo[2.2.2]octane (DABCO).

15. The method as claimed in claim 1, wherein the suitable solvent (A), solvent (B), solvent (C) or solvent (D) are independently selected from acetonitrile, acetic acid, acetone, hexane, heptane, octane, nonane, decane, dodecane, cyclopropane, cyclobutane, cyclopentane, cyclohexane, cycloheptane, cyclooctane, dimethyl formamide, ethylene dichloride, ethyl acetate, toluene, xylene, mesitylene, benzene, halogenated benzene, diisopropyl ether, t-butyl methyl ether, tetrahydrofuran, 2-methyltetrahydrofuran, dioxane, monoglyme, diglyme, methoxy-methane, methoxyethane, ethoxy-ethane, di-methoxyethane, di-ethoxyethane, dichloromethane, chloroform, dichloroethane, N,N-dimethylmethanamide, N,N-dimethylformamide (DMF), dimethylsulfoxide (DMSO), N-methyl-2-pyrrolidone (NMP), 1,3-dimethyl-3,4,5,6-tetrahydro-2(1H)-pyrimidinone, hexamethylphosphoramide, 1,3-dimethyl-2-imidazolidinone, or combinations thereof.

16. The method as claimed in claim 1, wherein the steps of said method step-a, step-b step-c and step-d are carried out at a temperature in a range of 0 to 150° C.

17. The method as claimed in claim 1, wherein
i. the suitable reagent in the step-a is selected from nitric acid (HNO₃), sulfuric acid, acetic acid, hydrochloric acid, or mixtures thereof;
ii. the suitable solvent (A) in the step-a is selected from dichloromethane, dichloroethane or acetonitrile;
iii, the step-a is carried out at a temperature in a range of 0 to 100° C.

18. The method as claimed in claim 1, wherein
i. the suitable base (I) in the step-b is selected from K₂CO₃, Na₂CO₃, NaOH, KOH or K₃PO₄;
ii. the suitable solvent (B) in the step-b is selected from dichloroethane, toluene, xylenes, chlorinated benzene, acetonitrile, dioxane, and dimethoxy ethane;
iii, the optional phase transfer catalyst is selected from tetraalkylammonium halide;
iv. the step-b is carried out at a temperature in a range of 25 to 130° C.

19. The method as claimed in claim 1, wherein
i. the hydrolyzing agent in the step-c is selected from an acid;
ii. the suitable solvent in the step-c is selected from acetic acid (AcOH), water or acetonitrile;
iii, the step-c is performed within a temperature range of 50 to 130° C.

20. The method as claimed in claim 2, wherein
i. the acid chloride in the step-d is selected from thionyl chloride (SOCl₂), mesityl chloride (MsCl), phosphorus pentachloride (PCl₅), phosphorus trichloride (PCl₃), oxalyl chloride, triphosgene or phosgene;
ii. the suitable solvent (C) and solvent (D) in the step-d are independently selected from dichloroethane, acetonitrile (ACN), N,N-dimethylformamide (DMF), ethyl acetate, pyridine, picoline, or mixtures thereof;
iii, the suitable base (II) and base (III) in the step-d are independently selected from alkali or alkaline earth metal carbonate, bicarbonate, hydroxide, hydrides or alkoxide, isopropyl amine, triethylamine, diisopropyl ethyl amine, triisopropyl amine, pyridine, picoline, N-methylmorpholine, N-methylpiperidine, N,N-(dimethylamino)pyridine (DMAP), lutidine, collidine, tetramethylammonium hydroxide, tetrabutylammonium hydroxide, 1,8-diazabicyclo[5.4.0]undec-7-ene (DBU), 1,4-diazabicyclo[2.2.2]octane (DABCO), 1,5,7-triazabicyclo[4.4.0]dec-5-ene (TBD), or 1,5-diazabicyclo[4.3.0]non-5-ene (DBN);
iv. the step-d is performed at a temperature within a range of 0 to 80° C.

21. The method as claimed in claim 10, wherein the suitable solvent (B) is selected from acetonitrile, acetic acid, acetone, hexane, heptane, octane, nonane, decane, dodecane, cyclopropane, cyclobutane, cyclopentane, cyclohexane, cycloheptane, cyclooctane, dimethyl formamide, ethylene dichloride, ethyl acetate, toluene, xylene, mesitylene, benzene, halogenated benzene, diisopropyl ether, t-butyl methyl ether, tetrahydrofuran, 2-methyltetrahydrofuran, dioxane, monoglyme, diglyme, methoxy-methane, methoxyethane, ethoxy-ethane, di-methoxyethane, di-ethoxyethane, dichloromethane, chloroform, dichloroethane, N,N-dimethylmethanamide, N,N-dimethylformamide (DMF), dimethylsulfoxide (DMSO), N-methyl-2-pyrrolidone (NMP), 1,3-dimethyl-3,4,5,6-tetrahydro-2(1H)-pyrimidinone, hexamethylphosphoramide, 1,3-dimethyl-2-imidazolidinone, or combinations thereof; the suitable base (I) is selected from an inorganic or organic base; the inorganic base can be selected from lithium hydrogen carbonate (LiHCO₃), sodium hydrogen carbonate (NaHCO₃), potassium hydrogen carbonate (KHCO₃), cesium hydrogen carbonate (CsHCO₃), sodium carbonate (Na₂CO₃), calcium carbonate (CaCO₃), cesium carbonate (Cs₂CO₃), lithium carbonate (LizCO₃), potassium carbonate (K₂CO₃), lithium hydroxide (LiOH), sodium hydroxide (NaOH), potassium hydroxide (KOH), cesium hydroxide (CsOH), calcium hydroxide (Ca(OH)₂), sodium diphosphate (Na₂HPO₄), sodium phosphate (Na₃PO₄), potassium diphosphate (K₂HPO₄), potassium phosphate (K₃PO₄), sodium fluoride (NaF), potassium fluoride (KF), cesium fluoride (CsF), lithium hydride (LiH), sodium hydride (NaH), potassium hydride (KH), sodium methoxide (NaOCH₃), sodium ethoxide (NaOCH₂CH₃), sodium tert-butoxide or potassium tert-butoxide; and the organic base can be selected from ethylamine, triethylamine, isopropylamine, diisopropylamine, triisopropylamine, pyridine, picoline, piperidine, methylmorpholine, N-methylpiperidine, N,N-(dimethylamino)pyridine (DMAP), lutidine, collidine, tetramethylammonium hydroxide, tetrabutylammonium hydroxide, choline hydroxide, 1,5,7-triazabicyclo[4.4.0]dec-5-ene (TBD), 2,3,4,6,7,8,9,10-octahydropyrimido-[1,2-a]azepine, 1,8-diazabicyclo[5.4.0]undec-7-ene (DBU), 1,5-diazabicyclo[4.3.0]non-5-ene (DBN), or 1,4-diazabicyclo[2.2.2]octane (DABCO).

22. The method as claimed in claim 10, wherein the step-b is carried out at a temperature in a range of 0 to 150° C.
